(19) United States
(12) Patent Application Publication
(10) Pub. No.: US 2018/0099140 A1
(43) Pub. Date: Apr. 12, 2018

(54) METHOD AND SYSTEM FOR TRIGGERING WOUND RECOVERY BY DELIVERING SOLUTION INTO THE PORES OF RECIPIENT

(71) Applicant: MICHAEL TAVGER, KATZRIN (IL)

(72) Inventor: MICHAEL TAVGER, KATZRIN (IL)

(21) Appl. No.: 15/837,046

(22) Filed: Dec. 11, 2017

Related U.S. Application Data
(63) Continuation-in-part of application No. 14/479,321, filed on Sep. 7, 2014.
Application No. 14/479,321 is a continuation-in-part of application No. PCT/IL2013/050369, filed on May 2, 2013.
(60) Provisional application No. 62/432,759, filed on Dec. 12, 2016.

(30) Foreign Application Priority Data
Jun. 18, 2012 (IL) 220486

(51) Int. Cl. A61N 1/30; A61M 37/00

(52) U.S. Cl. CPC A61N 1/303; A61M 2037/0007; A61M 37/00

(57) ABSTRACT

A method for delivering liquid into the pores of a recipient human skin is provided. The method includes delivering via a nozzle, at least one stream of the liquid under pressure to the pores, the stream having a cross-section with a diameter no greater than about 50 μm, applying a negative pressure across an area of the skin being treated, rotating the nozzle relative to the area at a speed up to about 200 revolutions per minute, contacting an electrode connected to a first terminal of a power source, having a first electric charge, to the skin, and contacting a second terminal of the power source, having a second electric charge, opposite the first electric charge, to the liquid, thereby imparting direct electrical current (DC) between about 5 μA and 0.1 mA to the skin in the vicinity of the pores. The delivering, applying, rotating, and contacting occur simultaneously.

### CROSS-REFERENCE TO RELATED APPLICATIONS

This application claims priority of U.S. Provisional Patent Application Ser. No. 62/432,759, filed Dec. 12, 2016, and is a Continuation-In-Part of U.S. patent application Ser. No. 14/479,321, filed Sep. 7, 2014, which is itself a Continuation-In-Part of International Patent Application No. PCT/IL2013/050369, filed May 2, 2013, which claims the benefit of priority from Israel Patent Application No. 220486, filed Jun. 18, 2012, the contents of which are incorporated by reference in their entirety.

### FIELD

This invention relates to systems and methods for providing conductive pathways through skin via solutions delivered into skin pores. In particular the invention relates to triggering recovery of wounds by electrical triggering of surrounding skin.

### BACKGROUND

Wounds generally heal naturally over time in a predictable manner. During recovery of acute wounds, a balance is maintained between production and degradation of molecules such as collagen which protect the damaged tissue until the wound is healed. Wounds which do not heal in a typical manner within about three months are considered to be chronic wounds and may take years to heal or in some cases may never heal at all and may remain at ongoing risk of infection.

Wounds may become chromic because their healing process is arrested at one of the phases of recovery. As a result the balance between production and degradation of collagen may be lost with degradation becoming dominant.

Various methods have been considered to restart the healing process in chronic wounds. Known treatments for chronic wounds include application of various dressings such as gauze or adhesive bandages, antimicrobial dressings, hydro-colloids, foam dressings. Alternatively negative pressure wound therapy or bioengineered skin may be used to treat an open wound.

However such methods involve treatment of the wound itself which seldom has positive results and often leads to new wounds, even greater damage and risk of infection.

There is a need for an effective treatment of chronic wounds which will accelerate their recovery. The invention addresses this need.

### SUMMARY

Accordingly the invention suggests a method and system for triggering wound recovery. It is a particular feature of the invention that unlike prior art treatments which treat the wound itself, embodiments of the method disclosed herein are directed to the treatment of the unaffected skin surrounding the wound.

Aspects of the invention provide conductive pathways through the skin surrounding the wound via solutions delivered into the skin pores. In further aspects, a potential difference is set up across the skin such that a direct electrical current may be passed therethrough skin's nerve terminals surrounding the wound to excite and trigger neurons of the central nervous system.

Such an electrical trigger may create a long-term phantom message of wounding and a consequent repair response resulting in the development of phagocytes, fibroblasts, angiogenesis and soft collagen creation.

It is therefore an object of the invention to facilitate the delivery of conductive solutions through the skin by infusion directly into the pores.

This object is realized according to the invention by a method and system having the features of the respective independent claims herein.

Therefore, according to another aspect of the presently disclosed subject matter, there is provided a method for delivering liquid into the pores of recipient human skin, the method comprising:
infusion of at least one jet of the liquid under pressure to the pores, the jet stream having a cross-section at its widest point smaller than an inlet opening of the pore;
contacting an electrode connected to a first terminal of a power source, having a first electric potential, to the skin; and
contacting a second terminal of the power source, having a second electric potential, opposite the first electric charge, to the liquid, thereby imparting a direct current (DC) electrical current through the pore by filling it with electro-conductive solution. Typically, the infusion and contacts occur simultaneously.

The method may further include the step of contacting a second terminal may include infusion liquid into pores in a region of the skin to which the second terminal is to be connected; applying a layer of electro-chemical gel to the region of the skin to which the second terminal is to be connected; and placing the second terminal in contact with the layer of electro-chemical gel.

Human skin has at least three types of skin appendages which could each be applicable for solution delivery, i.e., hair follicles, sebaceous glands and sweat glands. The proposed method of delivery of cosmetic and medical material exploits the fact that these skin appendages bypass stratum corneum defense providing access to the skin and underlying tissue. The modality of the method is that the device for medical solution delivery has a tip with one or more nozzles that are smaller than pore size. Negative pressure is applied in order to ensure that the outlets of the device nozzles dock with the inlets of the pores such that the jet is aligned with the pore. In order to deliver the material through a larger number of pores on the treated skin surface, the device is shifted relative to the pores along the external skin surface. When the outlets of the device nozzles coincide with the inlets of the pores, the delivery occurs.

Since the outlet diameter of the nozzle is smaller than the size of a pore opening, the pressure of the solution jet superposed with the pore does not meet any skin resistance and does not cause skin damage in the flow direction. The negative pressure causes the treated skin area to adjoin the inner surface of the nozzle tip. As the device is shifted over the skin surface the nozzle orifices and the pore inlets become connected for short periods of superposition and the pore channel is filled with the medicine or cosmetic solution. As a result of filling, the pore inlet is widened and filled with even more medicine or cosmetic. If the orifice of the nozzle were larger than that of the pore inlet, the jet pressure would also affect the skin region surrounding the pore, causing compressive deformation of the skin inward and consequently closing the pore and preventing the medicine or cosmetic from permeating into the skin. Moving the device over the skin surface allows for skin treatment by means of medical or cosmetic solution delivery to a large number of pores.

A device implementing the proposed method of medicine or cosmetic solution delivery into the human skin consists of a compressor, ejector, separation container (separator), container for medical or cosmetic solution storage, pumping unit, operational handpiece with a port for the solution supply under pressure and waste evacuating port.

Although pores occupy only a small portion of the entire skin surface, the total surface area of the interiors of the pores inside the skin can be as much as about 6 times greater than the rest of the outer epidermis surface. As the interior of the pores do not have exposure to sunlight and contact with atmospheric oxygen, pore duct epidermal coverage of two epithelial cells thereof is not strong. The presently disclosed subject matter may be used to facilitate the use of the macro-structure of the pores for transportation of medical or cosmetic solutions to a uniform depth over the treated skin surface.

According to one aspect of the presently disclosed subject matter, there is provided a method for delivering liquid into the pores of recipient human skin, the method comprising:
delivering, via a nozzle, at least one jet of the liquid medical substance under pressure into the pores, the jet having a cross-section with a diameter no greater than about 50 μm;
applying a negative pressure across an area of the skin being treated;
rotating the nozzle relative to the area at a speed ranging from 0 up to about 200 revolutions per minute;
contacting an electrode connected to a first terminal of a power source, having a first electric potential, to the skin; and
contacting a second terminal of the power source, having a second electric potential, opposite the first electric potential, to the electro-conductive liquid jet, thereby imparting a direct current (DC) electrical current through the pores by filling it with electro-conductive solution; wherein the delivering, applying, rotating, and contacting (i.e., the contacting of the first and second terminals of the power source) occur simultaneously. Where appropriate the direct electrical current imparted through the pores may be between 5 μA and 0.1 mA.

The first electric potential may be positive, with the second electric potential being negative such that a DC potential difference is maintained between the two electrodes.

The liquid may comprise a medical or a cosmetic solution.

According to a further aspect of the presently disclosed subject matter, there is provided a system for delivering a liquid, which may comprise a medical or cosmetic solution, into pores of a human's skin, the system comprising:
a storage container for containing the liquid;
a pumping unit coupled to the storage container for increasing the pressure of the liquid;
a compressed air ejector configured to generate a negative pressure;
a power source having first and second terminals with a potential difference maintained there between;
an electrode connected to the first terminal of the power source and being configured for being brought into contact with the skin during the delivery; and
a handpiece with a tip having at least one nozzle for delivery of the liquid into pores of the human skin wherein the nozzle delivers a liquid jet having a cross-section at its widest point smaller than an inlet of the pore, the handpiece being configured to bring the liquid into contact with the second terminal of the power source, thereby facilitating imparting a direct current (DC) electrical current through the pore by filling it with electro-conductive solution;

The electrical current may be between about 5 μA and 0.1 mA.

The diameter of the cross-section of the stream may be no greater than about 50 μm. The tip may be configured to rotate, for example at a speed up to about 200 revolutions per minute.

The suction mechanism may comprise a compressor and a gas ejector.

The handpiece may comprise a vacuum port connected to a vacuum unit configured for applying negative pressure via the tip of the handpiece across an area of skin being treated.

According to a further aspect of the presently disclosed subject matter, there is provided a system for delivering a liquid, which may comprise a medical or cosmetic solution, into pores of a human's skin, the system comprising:
a pumping unit coupled to the storage container for increasing the pressure of the liquid;
a power source having first and second terminals;
an electrode connected to the first terminal of the power source and being configured for being brought into contact with the skin during the delivery.
a handpiece comprising a tip having at least one nozzle for delivery of the liquid into pores of the human skin wherein the nozzle delivers a stream having a cross-section with a diameter no greater than about 50 μm, the handpiece being configured to bring the liquid into contact with the second terminal of the power source, thereby facilitating imparting an electrical current between 5 μA and 0.1 mA to the skin in the vicinity of the pores, the tip being configured to rotate at a speed up to about 200 revolutions per minute; and
a suction mechanism configured to generate a negative pressure via the tip.

The suction mechanism may comprise a compressor and a gas ejector.

The suction mechanism may comprise a vacuum port configured to be connected to a vacuum unit.

According to another aspect of the presently disclosed subject matter, there is provided a method for imparting DC electrical pulses across the skin. The method includes:
delivering an electro-conductive solution into the pores of a recipient human skin under treatment;
applying an electro-conductive gel onto a tip of an electrobrush, the electrobrush comprising a metal electrode and a silicon perforated tip;
allowing the electro-conductive gel solution to move through the perforated tip for making contact with the metal electrode;
allowing the electro-conductive gel to conductively couple with the solution within the pores thereby forming a conductive path from the metal electrode to the base of the pores;
attaching a body electrode to the skin area around treatment area for nerve activation, where required, in order to prepare a site for a body electrode to be attached, electro-conductive solution may be delivered into pores at the attachment site and an electro-conductive gel applied to the site so as to ensure a conductive path from the body electrode attachment site to the base of the pores; and
establishing an electrical circuit between the electrobrush and the body electrode through the nervous system situated therebetween and imparting direct electrical current (DC) to the skin through the solution.

According to another aspect of the presently disclosed subject matter, there is provided a system for delivering an electro-conductive solution into the pores of a recipient human skin under treatment. The system comprising:
a body electrode attached to skin area around treatment area for nerve activation;
the electro-conductive solution; and
an electrobrush, wherein the electrobrush comprises a metal electrode and a silicon perforated tip,
   wherein the solution flows on the perforated tip making contact with the metal electrode thereby closing an electrical circuit between the electrobrush and the body electrode through the nervous system situated therebetween and imparting direct electrical current (DC) to the skin through the solution.

The electrical current is between about 5 μA and 0.1 mA.

According to yet another aspect of the presently disclosed subject matter, there is provided an apparatus for delivering an electro-conductive solution into the pores of a recipient human skin under treatment. The apparatus comprising an electrobrush and a body electrode attached to skin area around treatment area for nerve activation. The electrobrush comprising:
a tip having perforations;
a metal electrode;
a body having three portions—a front portion, a middle portion, and a rear portion distal to the metal electrode;
a socket and;
a plug following the body,
   wherein the solution being applied onto the tip flows through the perforations thereof to contact the metal electrode, making a closed circuit with the body electrode through the nervous system situated therebetween and imparting direct electrical current (DC) to the skin through the solution.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1a illustrates one example of a system for the delivery of a medical or cosmetic solution to the skin via pores;

FIG. 1b illustrates another example of a system for the delivery of a medical or cosmetic solution to the skin via pores;

FIG. 2 is a partial section of an operational handpiece for use in the system shown in FIG. 1;

FIGS. 3a and 3b are enlarged schematic drawings showing a detail of the handpiece during delivery of medicine or cosmetic via human skin pores; and

FIG. 4 illustrates a method of delivering liquid into the pores of recipient human skin which may be carried out with the system illustrated in FIG. 1b.

FIG. 5 shows a schematic of another example of a system for the delivery of a medical or cosmetic solution via pores by applying electrical field via electro-conductive gel or cream;

FIG. 6 shows a method for applying electrical field via electro-conductive gel or cream which may be carried out with the system illustrated in FIG. 5; and

FIGS. 7A-7M illustrate exemplary skin problems before and after applying the methods of FIGS. 4 and 6 onto skin of the undergoing treatment.

### DETAILED DESCRIPTION

Reference will now be made in detail to embodiments of the invention, examples of which are illustrated in the accompanying drawings.

In the following detailed description, numerous specific details are set forth in order to provide a thorough understanding of the invention. However, it will be understood by those or ordinary skill in the art that the invention may be practiced without these specific details. In other instances, well known methods, procedures and/or components have not been described in detail so as not to obscure the invention.

The invention will be more clearly understood from the following description of the methods thereof, given by way of example only with reference to the accompanying drawings. In the descriptions that follow, like numerals represent like elements in all figures. For example, where the numeral (2) is used to refer to a particular element in one figure, the numeral (2) appearing in any other figure refers to the same element.

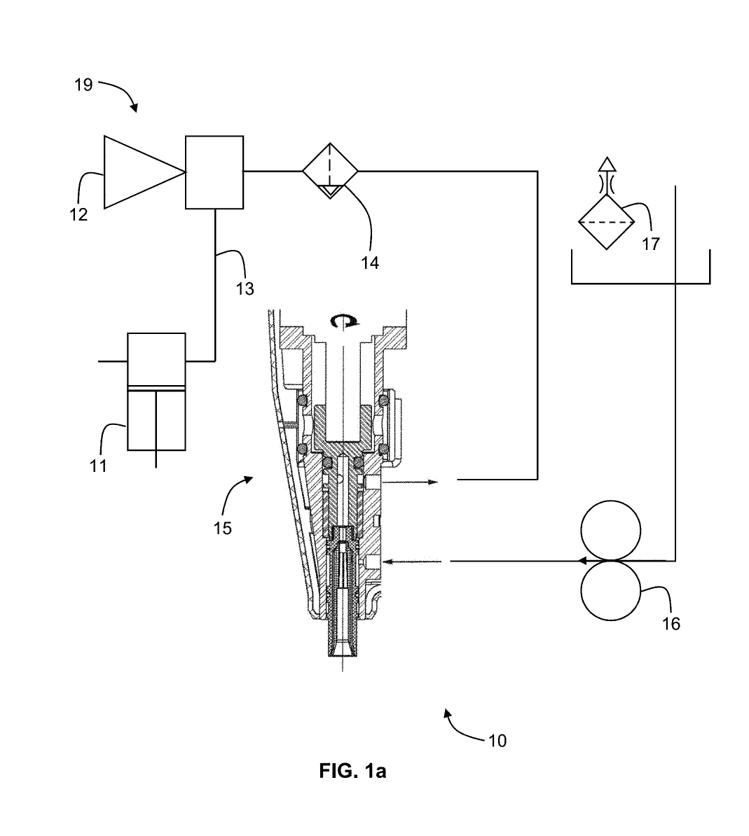

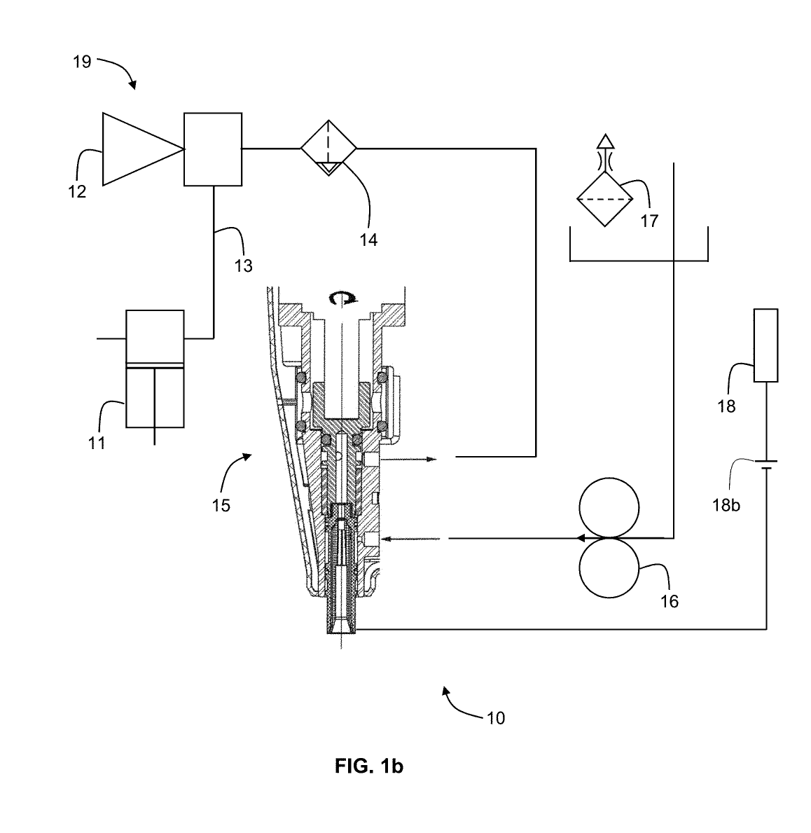

Referring to FIG. 1a, there is shown one example of a system 10 comprising suction mechanism, which is generally indicated at 19, connected to a separation container 14 which is connected to an operating handpiece 15. The handpiece 15 is connected to a pumping unit 16 which is in turn connected to a storage container 17 for storing a liquid such as a medical or cosmetic solution. According to some examples, for example as indicated in FIG. 1a, the suction mechanism may comprise a compressor 11 connected to a gas ejector 12 (which is connected to the separation container 14) by a tube 13. According to other examples (not illustrated), the suction mechanism 19 comprises a vacuum pump or any other suitable arrangement, connected to a vacuum port (not illustrated) in the handpiece 15.

The compressor 11 is used for the air compression needed to generate negative pressure in the gas ejector 12. The gas ejector 12 does not contain moving parts, which makes it preferable for negative pressure generation in humid media. The separation container 14 provides liquid separation of the liquid-gas mixture. The storage container 17 is used for solution storage and its delivery to the pumping unit 16, which provides the high pressure used for the solution delivery.

FIG. 1b illustrates another example of a system 110, which is a modification of the system 10 illustrated in FIG. 1a. The system 110 comprises the same components as described above with reference to the system illustrated in FIG. 1a, and operates similarly thereto. In addition, the system 110 illustrated in FIG. 1b comprises an electrode 18. The electrode 18 is connected to one terminal (i.e., either positive or negative) or a power source 18b, which may be any suitable source of electrical power. The other terminal (having the opposite potential) of the power source is connected to part of the handpiece 15 with which the liquid therein comes into contact, such as the tip 23 (described below). The electrode 18 is designed to contact a patient's skin during use, for example by being grasped thereby or by being clasped/fastened thereto. Thus, the patient completes a circuit between the two terminals of the power source 18b, facilitating an electrical current from being imparted to his skin in general, and in the vicinity of the pores in particular.

It will be appreciated that while the present disclosure describes, by way of non-limiting example only, that the electrode connects the user's skin to the positive terminal of the power source 18b, and the handpiece 15 connects the liquid to the negative terminal of the power source, the reverse may be the case, i.e., the electrode may connect the user's skin to the negative terminal of the power source, while a the handpiece connects the liquid to the positive terminal of the power source.

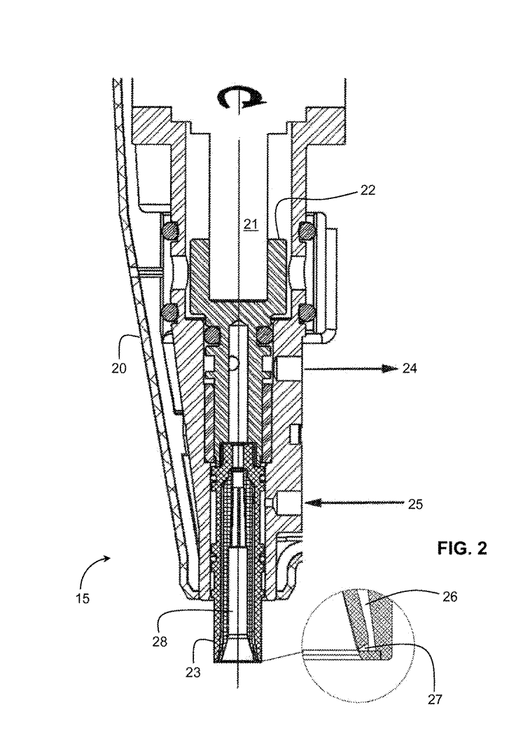

FIG. 2 shows in enlarged scale that the operating handpiece 15 includes an outer casing 20 accommodating a motor 21, which is coupled to a rotary drive 22 that is rotatably coupled to a tip 23. A suction port 24 in the casing 20 is used for applying the negative pressure inside the handpiece 15 and for the evacuation of waste into the separation container 14. A supply port 25 in the casing 20 is used to supply solution under pressure from the pumping unit 16 to the tip 23. The tip 23 comprises two assembled parts which form the unit with two channels. An external channel 26 is used to supply the cosmetic or medical solution under pressure via the supply port 25 to one or more nozzles 27 positioned in the distal part of the tip 23, which contact the skin surface. The nozzles 27 face toward the longitudinal axis of the tip 23 and perpendicular to its inside surface. An internal channel 28 of the tip 23 is designated for the waste evacuation via the suction port 24 and for gripping the treated skin area.

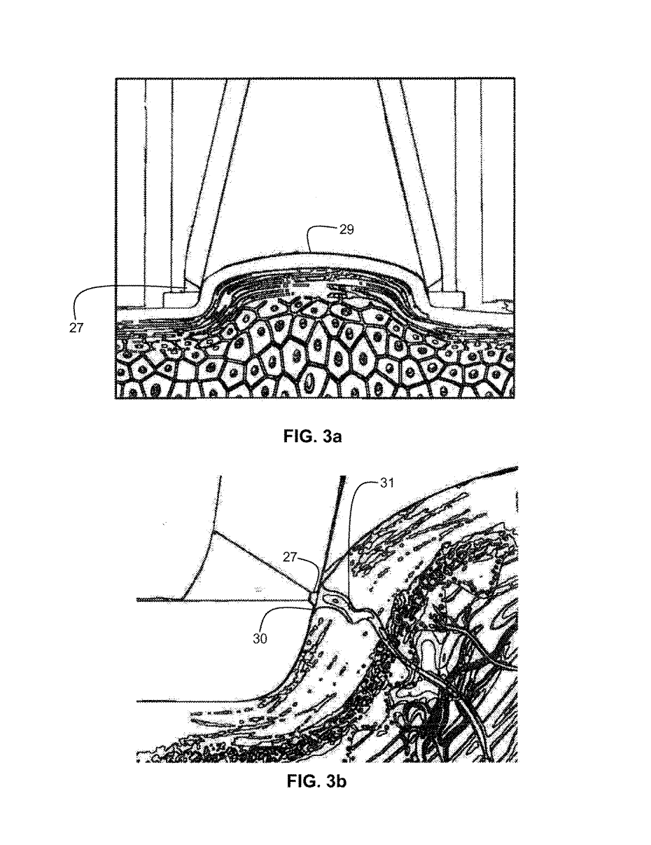

The operation of the system 10 illustrated in FIG. 1a will now be described. First the compressor 11 is activated, which applies air under high pressure to the ejector 12. The ejector 12 generates negative pressure, i.e., suction, in the separation container 14 and the handpiece 15. When the distal part of the tip 23 of the handpiece 15 contacts the skin surface, limited deformation of the skin region 29 within the perimeter of the tip 23 occurs. Owing to the negative pressure, the skin is drawn into the open chamber 28 of the tip 23 to a level that overlaps the nozzles 27 and is seated along the internal contour of the tip 23, thereby adopting its shape 29 along the points of contact as shown in FIG. 3a. The pumping unit 16 is then activated which supplies the liquid under pressure to the handpiece 15 via the supply port 25 from the storage container 17. At the same time, the motor 21 is activated and starts to rotate the tip 23 via the rotary drive 22. The medical or cosmetic solution ejected by the nozzles 27 of the rotating tip 23 flow to the skin surface 29 (shown in FIGS. 3a and 3b) located inside the open chamber 28 of the tip 23.

A jet of the medical or cosmetic solution under pressure (of about 1-2 bar) is delivered via the nozzles 27 and circulates over the deformed skin region (shown in FIG. 3a) during rotating contact of the tip 23. At the moments when, as shown in FIG. 3b, the outlet of a nozzle 27 in the tip 23 is located wholly within an inlet 30 of a pore, sluicing occurs, whereby the channel of the nozzle 27 is momentarily connected to the pore channel 31. Since the exit diameter of the nozzles 27 is smaller than that of the pore inlet 30, the liquid jet permeates into the pore interior without any resistance. Thus, during coincidence of the nozzles 27 and the pore inlet 30, the pore channel 31 is filled with the medical or cosmetic solution.

Pores contain structured hydrophilic and lipophilic domains with unilateral conductivity only toward the outside. However they are not protected against propagation of a single jet of solution flowing centrally toward the channel depth. During coincidence between the nozzle and the pore inlet, the pore channel is filled with a certain amount of the liquid. The pore channel 31 continues filling as long as the moving nozzles 27 of the tip 23 and the pore inlets 30 remain coincident. The amount of liquid permeating into the pores depends on the difference between the size of the solution delivery nozzle 27 and that of the pore inlets 30, i.e. as the pore size increases so too does the sluicing time with the nozzle. On the other hand the faster the tip rotation velocity, the shorter the sluicing duration. As the pressure of the medical or other solution rises, the quantity of liquid entering the pore increases. When motion of the orifice of the nozzle 27 overlaying the pores inlet 8 is arrested, the stretched throat of the pore contracts and encapsulates the residual solution in the pore channel 31. Treatment is performed by moving the handpiece 15 over the skin surface. When used medically, it has been demonstrated experimentally that a tip 23 with an outer diameter of 5 mm with a nozzle diameter of 50 μm provided the best medical effect. The optimal rotation velocity of the tip 23 was found to be 160 revolutions per minute (RPM) with a solution pressure of 1-6 bars. According to some examples, the rotational velocity for certain applications may be different, for example up to 200 RPM.

The average liquid volume permeating inside an individual pore depends on jet pressure, the rotation velocity of the tip 23 and its geometry, and should reach 0.05 -0.3 mm³. The maximum estimated amount of liquid spread over all the affected skin appendages is about 2 ml/200 cm².

For example in the case of an aesthetic procedure, the delivery of mild Alpha Hydroxy Acids (2-5%) via the pores extends to the depth of the dermis without damaging the skin. Usually this is possible only if skin integrity has been disrupted, i.e. the epidermis and dermis are damaged. In response to what appears as a threat, namely penetration of foreign solutions into the skin, the immune and nervous systems activate to repair potential damage from what is perceived as possible irritant penetration inside the skin. Thus, a real inflammatory response is generated by the extremely mild irritants that have permeated through the pores. The body's natural immune and infection protection systems promptly go into action to heal the non-existent damage to the skin.

As a result cell multiplication rate is increased and new epidermis cells are generated. The gross factors responsible for natural collagen production are activated in the dermis.

The method in the aesthetic application case, cosmetic solution delivery via pores under pressure, causes thickening of the dermis. Production of additional natural collagen improves skin density and causes smoothing of small wrinkles. As a result of sufficiently deep penetration into the dermis, a process of neurocosmetic and regenerative effect on the skin is steadily developed and persists for months. The delivery of non-damaging solutions via pores enables all the natural functions of skin protection and interaction with the environment.

Operation of the system 110 as described above with reference to FIG. 1b is similar to the operation of the system 10 of FIG. 1a, with the addition of an application of an electrical current to the patient and the liquid delivered by the system 110. In order to accomplish this, the electrode 18 is brought into contact with the patient's skin, for example by having the patient grasp it or by fastening it around a part of his body, such as the wrist, finger, shoulder, etc. Thus, the patient is connected to, e.g., the positive terminal of the power source 18b. An electrical current is thereby imparted to the skin, in particular in the vicinity of the pores. The current may be about 0.25 mA. As the diameter of the stream is about 50 μm, the current density is about 130 A/cm². This difference in charge between the patient's skin and the liquid dispensed via the handpiece facilitates delivery of the liquid through the pores.

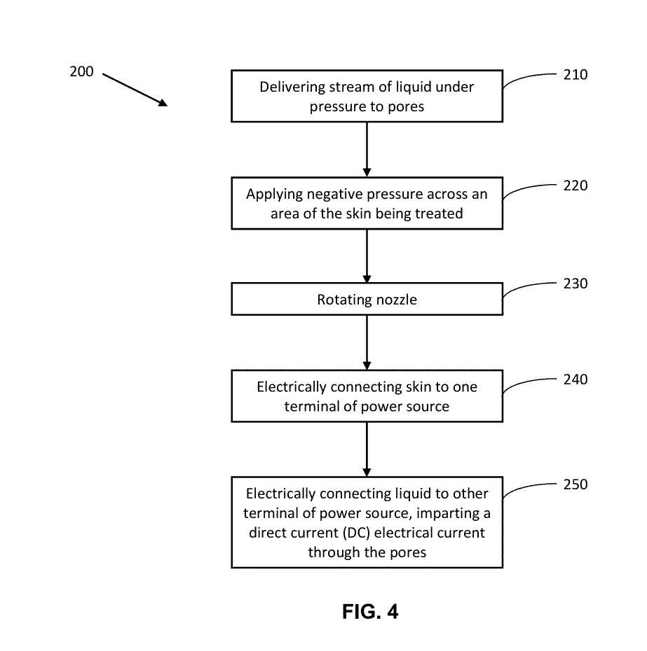

As illustrated in FIG. 4, the system 110 may be used in a method 200 to deliver the liquid into the pores of recipient human skin. In step 210, a stream of the liquid is delivered to the pores under pressure. The stream may have a cross-section which, at its widest point, is smaller than an inlet opening of the pore. For example, the cross-section may have a diameter which is no greater than about 50 μm.

In step 220, a negative pressure is applied across the area of the skin being treated.

In step 230, the nozzle via which the stream is delivered is rotated. The speed of the rotation may be up to about 200 RPM. According to some examples, it is between about 160 RPM and 200 RPM.

In step 240, the skin is electrically connected to one terminal of the power source 18b, for example via the electrode 18.

In step 250, the liquid is electrically connected to the other terminal of the power source 18b. This imparts an electrical current to the skin in the vicinity of the pores. This current may be in the range of 5 μA and 0.1 mA.

It will be appreciated that some of the steps of the method, for example applying a negative pressure (step 220) and/or rotating the nozzle (step 230) may be optional.

It will be further appreciated that all steps of the method may be carried out simultaneously, e.g., such that the delivering of the liquid happens at the same time that the negative pressure is applied, the nozzle is rotated, and the terminals of the power source 18b are connected to the skin and/or liquid.

It is to be understood that while the invention has been described with particular regard to the injection of a medicinal solution through the pores of a human patient, it is equally applicable to the injection of non-medical solutions such as cosmetics in non-therapeutic treatment of the human body.

It is also to be understood that typically the nozzles are circular in cross-section, such that the nozzle diameter is less than the inlet size of the pores. However, the nozzles need not be circular in cross-section provided that their outlet produces a stream whose cross-section at its widest point is smaller than the narrowest point in the inlet opening of a human skin pore.

It has been found that the system and method described above may have surprisingly uses producing and unexpected results when applied to the skin of subjects.

Because the pore delivery system both generates micro streams of solution and anchors these micro-streams to the skin surface, solutions may be infused into skin appendages such as sweat glands, follicle ducts and the like.

Accordingly, the inner surface of pore ducts, sweat glands and follicles may be used for the application and delivery of solutions for aesthetic treatments.

Although these inner surfaces point inward towards the skin's dermal depth, they remain a part of external epidermis. The total area of these inner surfaces is six times greater than that of the visible skin surfaces. Moreover, the inner surfaces provide direct contact with surrounding living tissue. Thus it has been found that the inner surfaces may be a target for treatment of the aesthetic properties and health of skin and underlying tissue.

The delivery system has been found to be a highly effective method for efficiently treating all types of skin. The delivery system does not inflict any damage to the skin therefore bypassing many of the shortcomings typical of other skin treatment devices. Delivering solutions directly into the pores, the method may infuse and trap minute droplets of solution inside the epidermal appendages (pores) to reach the dermis that lies behind the duct walls.

By penetrating the pore cavities, the system extends the treatment of skin beyond the outer surface of the skin and into the surrounding dermis area. Because the solution is trapped within the core cavity, a selected solution may be applied to the large absorption area of the inner surfaces of each pore duct. Furthermore, surprisingly, the prolonged entrapment of the solution has been found to provide a prolonged action time during which the solution may deliver the treatment.

The pore delivery system features a unique nozzle tip, having a diameter of 50 μm. Such a nozzle tip, which may be disposable may generate a tiny liquid solution jet to infuse active ingredients directly into the pores. For better contact between the tip and the skin surface, the system further applies negative pressure, which attracts the skin's surface to the rotating nozzle tip. While the operator guides the delivery device over the treatment area, the nozzle tip is briefly in contact with open pore orifices, which allows the solution to flow from the nozzle under moderate pressure into the epidermal appendage ducts, filling them with solution.

By maintaining an electrical potential difference between a first electrode in the device and a second electrode in contact with the subject, the infused solution may further serve as electroconductive media conducting direct electrical current into tissue beyond the usually electrically insulated skin via the filled cavities of the ducts, pores or other skin appendages. The conductive solution allows a power source inside the operation handpiece to generate an electrical current through the skin appendages and into the tissue interior.

In order to generate the direct current, it may be required to attach the second electrode (sometimes referred to as ‘the body electrode’) to an external skin surface. It is noted that in order to ensure electrical contact between the body electrode and the subject, before attaching the body electrode to the skin of the subject, the skin onto which the body electrode is to be attached should itself be treated with the pore delivery system so as to provide conductive pathways into the tissue beneath the external skin surface of the target attachment site.

Flow of electrical current through the solution inside the pore may further cause a diathermia effect due to high electrical density and small (50μ) pore's diameter. Rising of temperature can be significant but the duration of electrical current may be extremely short so as not to harm the skin but only to activate Heat shock proteins (HSP) and neuro proteins. Such proteins are produced by cells in response to exposure to stressful conditions. It has been found that such mild stress-induced stimulation of heat-shock protein synthesis accelerates generation of fibroblasts for growing a new connective tissue enriched with collagen and elastin.

Accordingly, wound healing may be accelerated by a non-invasive and non-wounding treatment of sites adjacent to an extant wound. It is particularly noted that this accelerated is generated without any treatment of the wound itself but by producing a phantom wound in proximity to the extant wound. The phantom wound may be generated by applying selected chemical reagents and/or electrical currents to skin appendages in the vicinity and surroundings of the extant wound.

The total inner pore duct-wall surface area can be six times greater than the outer skin surface area. This interior area is in direct contact with the surrounding dermis responsible for the skin's main aesthetic properties: strength, elasticity, appearance, freshness, vigor and youth.

Short DC electricity activation of skin appendages may cure problems of aging which are not due to disease but more subtle changes in neurobiological systems, including muscle weakening, loss of reproductive functions, changes in body weight, etc.

Further the treatment may provide methods that can stop or reverse the slow degeneration of distal axons that is common to most peripheral neuropathies.

In contradistinction to traditional neurology treatment and which use high-frequency alternating current (AC) and electromagnetic field to induce AC electrical currents in the tissue beneath the skin, the methods described herein may provide DC currents with both polarities or pulses to the area around the wound directly via conductive pathways through the pores.

Most nerve cells situated in the epidermis, skin dermis and underlying tissue respond to DC current more specifically than to high frequency current. The DC electricity reaches the nerve fibers from skin surface through the bypassing skin appendages interacts and spreads along nerve fibers helping to reactivate its conductive and sensory function. Changing the polarity of electricity allows the restoring of informative and controlling function of axons. Due to the very short duration of an electrical current via the skin appendage through rotating miniature jets of solution the result is continuous and forms by using the device, applied with silicon single use tip. The construction protects the patients skin from direct contact with a metal electrode, insulated perforated by silicon tip. The electrical simulation may require at least two batteries of 9V totaling 18V.

The same electrical potential may be applied via electroconductive gel or cream, providing contact between the skins pores already filled with solution and electrode of a device. Shifting of a device along the previously treated skin areas operator can continue DC Trans Cutaneous Stimulation treatment. Changing voltage, polarity and placement area of body electrode the system can treat most hard-to-repair skin conditions, such as looseness, muscle aging and myopathies, improving its tightening and making lifting.

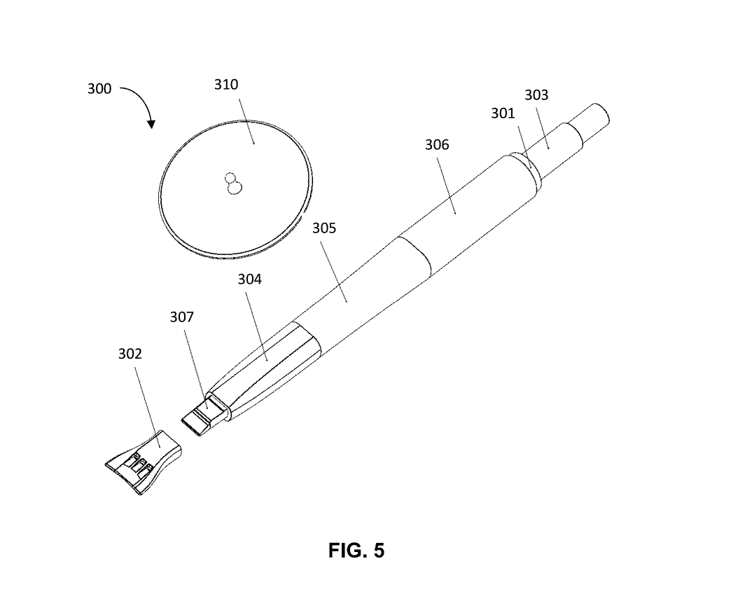

In some embodiments, an electrobrush 300 (FIG. 5) may be provided to deliver electroconductive solution into skin pores. The electrobrush 300 may further include a tip 302 having at least one perforation. In some embodiments, the tip 302 may be a brush or a hard surface. The tip 302 may be configured such that the gel/cream can be applied thereon. The gel or cream may be any electroconductive gel/cream that is already known in the art and used for various other applications such as ECG or Ultrasound. Examples of the gel may include such as but are not limited to Spectra 360, TAC gel, and so on.

The tip 302 may further be attached to a metal electrode 307. Examples of metals for making electrode may include, but are not limited to, stainless steel, aluminum, tungsten, platinum, and so on. The electrode 307 may also be made from any material that can conduct electricity like alloys. The gel or cream when applied onto the tip 302 manually may flow into the perforations of the tip 302 and may further come into contact with the electrode 307. In some embodiments, the tip 302 may be made from biomaterials such as including but are not limited to silicon, titanium, and so on. In some embodiments, a silicon cap may be applied onto the electrode 307, insulating the electrode from direct contact with the skin thereby preventing the shock. The electrobrush 300 may further include a body having three portions—a front portion 304, a middle portion 305, and a rear portion 306. The body may have any shape such as circular, ovular, cylindrical, and so on. In some embodiments, the front portion 304 may be tapered and have diameter greater than the electrode 307 such that the electrode 307 fit well into the front portion 304. In some embodiments, the body may have increasing diameter from the front portion 304 to the middle portion 305 and further to the rear portion 306. A socket 301 circumvented around a plug 303 may be attached distal to the rear portion 306. The socket 301 may have diameter smaller than that of the rear portion 306.

In some embodiments, a body electrode 310 may be attached onto skin area near or around to the skin area to be treated. The body electrode 310 is well known in the art and may be configured such that a closed circuit may be formed between the gel/cream attached to the electrode 307 and the body electrode 310 with nerve system situated therebetween, activating the nerve cells and imparting direct electric current (DC) through the gel/cream to be delivered into the skin pores. In some embodiments, the electrobrush 300 may be applied to the skin area wherein the skin pores are once treated and filled with the liquid through the method discussed hereinabove in FIG. 4.

In some embodiments, an apparatus, a system and a method thereof may be disclosed herein utilizing the electrobrush 300 and the body electrode 310, already discussed in detail hereinabove. The system may also include a storage container for containing the gel/cream.

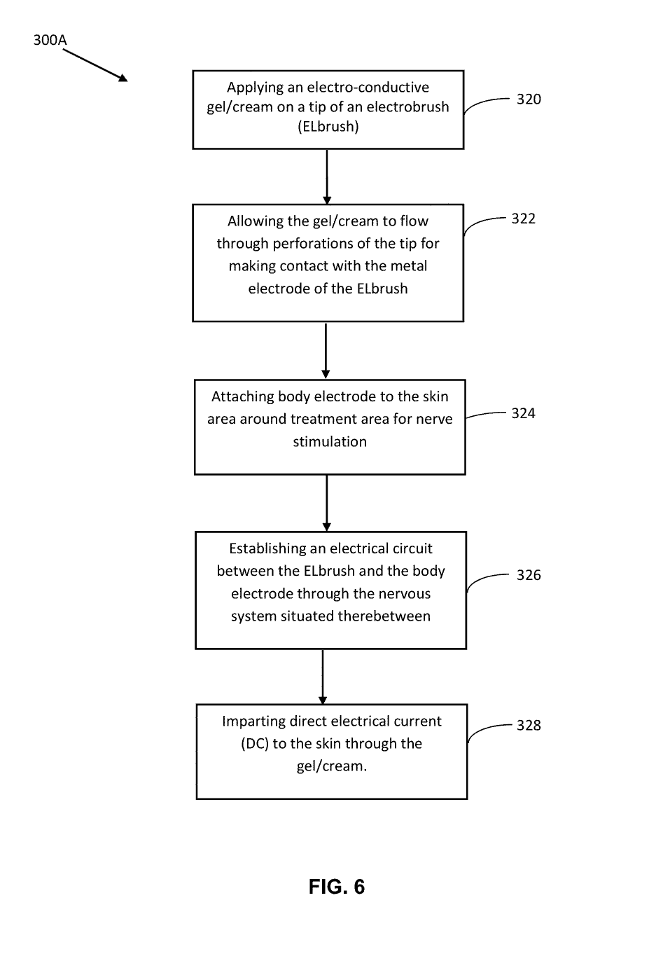

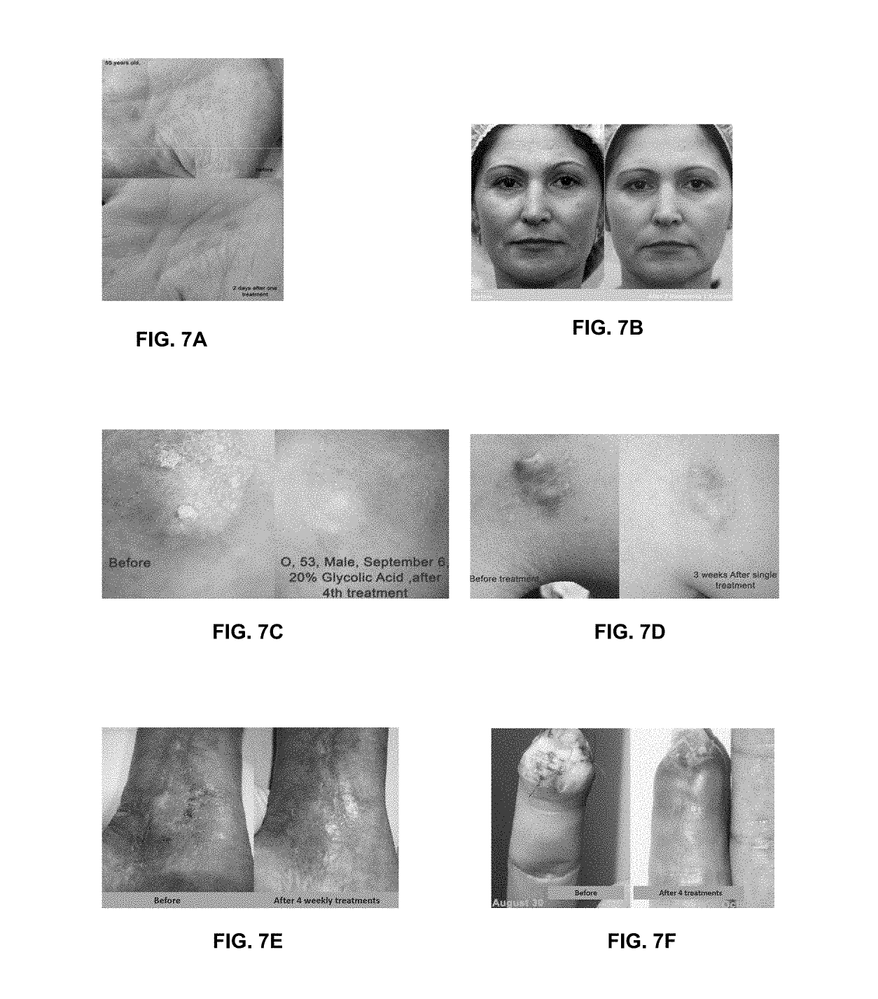

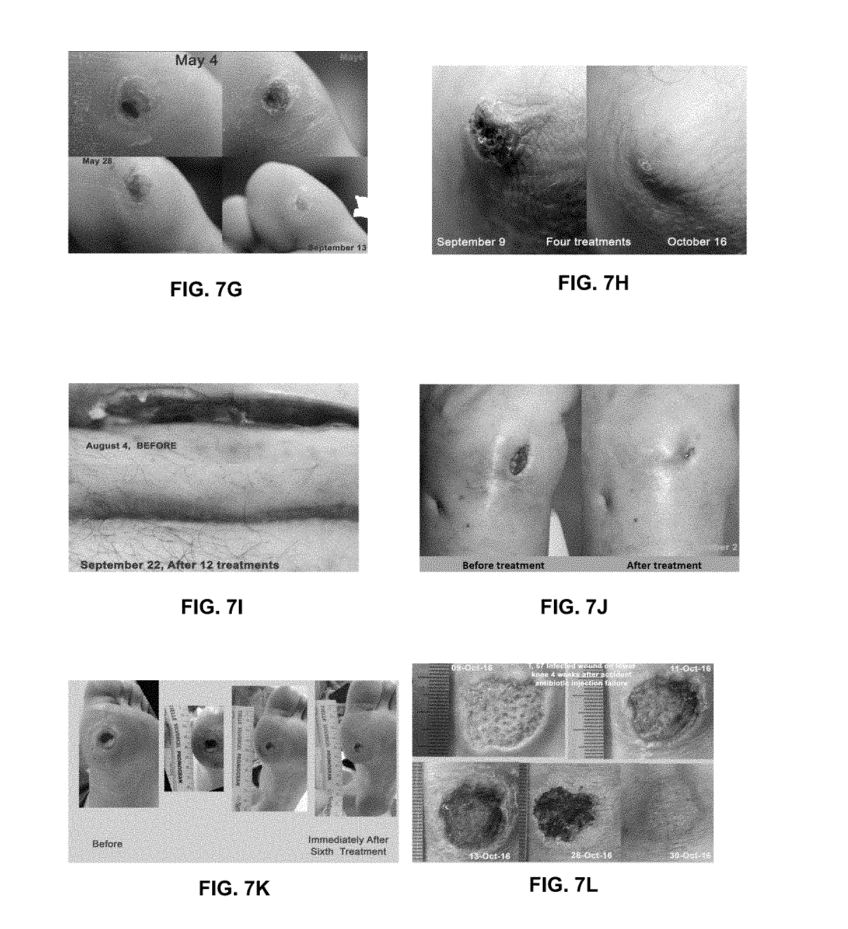

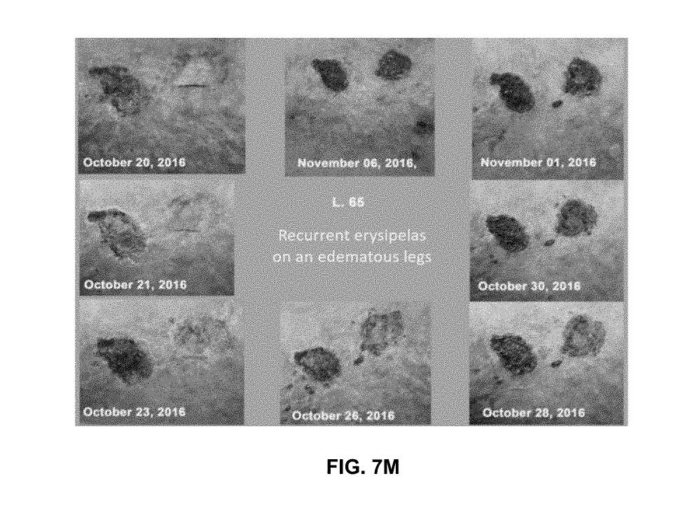

In some embodiments, a method 300A as shown in FIG. 6 may be disclosed for delivering the electroconductive gel/cream into the pores of recipient human skin. Sequence of the steps of the method 300A disclosed herein may be exemplary for the sake of understanding the skill in the art. The method 300A may include applying the gel/cream onto the tip of the electrobrush at step 320. The gel/cream may further be allowed to flow into perforations of the tip and contacting the metal electrode at step 322. The method 300A may further include attaching the body electrode to the skin area around the skin to be treated at step 324. The body electrode and the gel/cream onto the metal electrode makes a closed circuit with nerve system attached therebetween at step 326. Therefore, a direct electrical current (DC) may be imparted through the gel/cream to the skin pores at step 328.

In particular, it is noted that the delivery system may be used to fill ducts of skin appendages with various solutions for different aesthetic indications. Such treatment could be used alongside more typical applying means. Notably, the delivery system can be used to deliver solutions to the inner surfaces of the pore ducts, thereby treating some of the most difficult skin conditions, such as Melasma, Scars, Looseness, Pigmentation, Muscle Aging and Myopathies, Vascular smooth muscle disorders, Mature Collagen Remodeling and the like.

Various treatment methods and formulations for selected application in aesthetic medicines are presented in the table 01 below.

TABLE 01

| | Conditions | Contents | Procedure |
|---|---|---|---|
| Middle-aged | Anti-Aging | 1% NaCl | TIP ROTATION 100% no electricity |
| | | 5% Ascorbic Acid | TIP ROTATION 60% +electricity |
| | | 5-10% Lactic Acid | level 2 |
| | | | Elbrush (+electricity level 2) |
| Antiaging | Anti-Aging | 1% NaCl | TIP ROTATION 100% no electricity |
| | Lifting | 5% Ascorbic Acid | TIP ROTATION 60% +electricity |
| | | 25% Lactic Acid | level 3 |
| | | 5% Glycolic Acid | Elbrush (+electricity level 3) |
| | | | 360° around neck |
| Baldness | Hair | 1% NaCl | Treat only problematic areas |
| | Body's Scars | 5% Ascorbic Acid | TIP ROTATION 60% +electricity |
| | Tightening | 30% Glycolic Acid | Level 1 |
| | | | Elbrush (+electricity 1-2) |
| Problem areas | Pigmentation, | 3% NaCl | TIP ROTATION 100% no electricity |
| of Skin | Acne, Oily skin, | 5% Ascorbic Acid | TIP ROTATION 60% −electricity |
| | Seborrhea, | 0.5% Salicylic Acid | level 2 |
| | Melasma | | Elbrush (−electricity level 2-3) |
| | Areal treatment | | |
| | Face Scars | 1% NaCl | TIP ROTATION 100% no electricity |
| | Problematic areas | 5% Ascorbic Acid | TIP ROTATION 60% +electricity |
| | treatment | 0.5% Salicylic Acid | level 3 |
| | | 8-10% Lactic Acid | Elbrush (+electricity level 3) |
| | Cleansing Lotion | 1% NaCl | Removes from skin oil, |
| | AirBrush | 3% Citric Acid | contamination to protect and save |
| | | 5% Glycolic Acid | the TIPs. Relaxing action after |
| | | | laser, chemical peeling RF and etc. |

It has further been found that the pore delivery system and method may have unexpected success in the acceleration of the healing of skin wounds and the treatment of chronic wounds in general.

Infusion of chemical reagents into epidermal appendages and their deposition upon the inner surfaces of the ducts naturally penetrates the skin's anatomical barriers—physical, chemical and biological. When an intraductal infusion of an active solution reaches the inner epithelial surface, the local acute bio-interaction process may begin to generate the healing response which triggers a wound healing process. The infused solution may become retained within the follicular duct in the depths of the dermis, providing both a larger potential absorption area and a longer contact time. This magnifies the biological activity of the active ingredients in the solution. Furthermore, this may also create a long-term phantom message of wounding thereby activating the body's natural repair response. Accordingly, the body may respond by inducing the development of phagocytes, fibroblasts, angiogenesis and soft collagen creation.

The disclosed systems and methods have been reported successfully treated variety of skin problems as shown in FIGS. 5A-5M. FIG. 5A shows skin of a 55 year old patient having eczema being improved after 2 days utilizing the above disclosed systems, methods, and the apparatus. FIG. 5B shows skin of face of a female suffering from melasma having completely treated after 3 days of the treatment using the above disclosed systems, methods, and the apparatus. FIG. 3C shows psoriasis in 53 year old male being treated after 4^{th} treatment. FIG. 3D shows lightening of a postoperative hypertrophic keloid scar developed after removal of atheroma 2 years ago after 3 weeks of the treatment. FIG. 3E shows wound healing acceleration in 66 years old after a 4 weekly treatment. FIG. 3F shows treatment of contamination due to amputation following industrial trauma after 4 treatments. FIG. 3G shows another example of wound healing acceleration of an 8 month old non-healing callus wound 8 mm deep in a big toe. FIG. 3H shows healing of a 2 year old trauma wound after four treatments. FIG. 3I shows treatment of complications arose following urological surgery after 12 treatments. FIG. 3J shows treatment of a knee trauma after utilizing the above disclosed systems, methods, and the apparatus. FIG. 3K shows treatment of two years old (not healing) wound due to diabetics after sixth treatment. FIG. 3L shows wound healing acceleration of an infected wound on lower knee 4 weeks after antibiotic injection failure and being treated after 4 treatments. FIG. 3M shows wound healing acceleration of recurrent erysipelas on edematous legs after six treatments.

Those skilled in the art to which this invention pertains will readily appreciate that numerous changes, variations and modifications can be made without departing from the scope of the invention mutatis mutandis.

While embodiments of the invention and its advantages have been disclosed in the above Detailed Description, the invention is not limited thereto, but only by the scope of the appended claims.

What is claimed is:

1. A method for delivering liquid into pores of a recipient skin, the method comprising:
delivering, via a nozzle, at least one stream of said liquid under pressure to said pores, said stream having a cross-section with a diameter no greater than 50 micrometers;
applying a negative pressure across an area of the skin being treated;
rotating said nozzle relative to the area at a speed up to about 200 revolutions per minute;
contacting an electrode connected to a first terminal of a power source, to the skin; and
contacting a second terminal of said power source, to said liquid; and
imparting a potential difference between said first terminal and said second terminal such that a direct electrical current (DC) is generated of between 5 microamperes and 0.1 milliamperes through the skin in the vicinity of said pores,
wherein said delivering, applying, rotating, contacting and imparting occur simultaneously.

2. The method according to claim 1, wherein said liquid comprises a medical or a cosmetic solution.

3. A method for delivering liquid into pores of a recipient human skin, the method comprising:
delivering at least one stream of said liquid under pressure to said pores, said stream having a cross-section at its widest point smaller than an inlet opening of said pore;
contacting an electrode connected to a first terminal of a power source, to the skin; and
contacting a second terminal of said power source, to said liquid; and
imparting a potential difference between said first terminal and said second terminal such that a direct electrical current (DC) is generated through the skin in the vicinity of said pores,
wherein said delivering and contacting occur simultaneously.

4. The method according to claim 3, wherein the step of contacting a second terminal comprises:
delivering liquid into pores in a region of the skin to which the second terminal is to be connected;
applying a layer of electro-conductive gel to the region of the skin to which the second terminal is to be connected; and
placing said second terminal in contact with said layer of electro-conductive gel.

5. The method according to claim 3, wherein the diameter of the cross-section of said stream is no greater than 50 μm.

6. The method according to claim 3 further comprising applying, simultaneously with said delivering and contacting, negative pressure across an area of the skin being treated.

7. The method according to claim 3, wherein said liquid stream is delivered via a nozzle, the method further comprising moving said nozzle relative to said area of the skin.

8. The method according to claim 7, wherein moving said nozzle comprises rotating the nozzle.

9. The method according to claim 8, wherein said nozzle is rotated at a speed no greater than 200 revolutions per minute.

10. The method of claim 3 for imparting DC electrical pulses across the skin, the method comprising:
delivering the electro-conductive solution into the pores of the recipient skin;
applying an electro-conductive gel onto a tip of an electrobrush, wherein the electrobrush comprises a metal electrode and a silicon perforated tip;
allowing the electro-conductive gel to move through the silicon perforated tip for making contact with the metal electrode;
allowing the electro-conductive gel to conductively couple with the solution within the pores thereby forming a conductive path from the metal electrode to the base of the pores;
attaching a body electrode to the skin area around treatment area for nerve activation; and
establishing an electrical circuit between the electrobrush and the body electrode through the nervous system situated therebetween and imparting said potential difference through the electro-conductive solution.

11. The method of claim 3, wherein the said electrical current is between 5 μA and 0.1 mA.

12. A system for delivering liquid into pores of a human skin, the system comprising:
a storage container for containing said liquid;
a pumping unit coupled to the storage container for increasing pressure of said liquid;
a suction mechanism configured to generate a negative pressure;
a power source having a first terminal and a second terminal;
an electrode connected to the first terminal of said power source and being configured to brought into contact with the skin during the delivery of liquid; and
a handpiece with a tip having at least one nozzle for delivery of the liquid into pores of the human skin wherein said nozzle delivers a stream having a cross-section smaller than an inlet of said pores, said handpiece being configured to bring the liquid into contact with the second terminal of the power source and imparting a potential difference between said first terminal and said second terminal such that a direct electrical current (DC) is generated through the skin in the vicinity of said pores.

13. The system according to claim 12, wherein said direct electrical current is between 5 μA and 0.1 mA.

14. The system according to claim 12, wherein the diameter of the cross-section of said stream is no greater than about 50 μm.

15. The system according to claim 12, wherein said tip is configured to rotate.

16. The system according to claim 12, wherein said tip is configured to rotate at a speed no greater than 200 revolutions per minute.

17. The system according to claim 12, wherein said suction mechanism comprises a compressor and a gas ejector.

18. The system according to claim 12, wherein the handpiece comprises a vacuum port connected to a vacuum unit configured for applying negative pressure via the tip of the handpiece across an area of skin being treated.

19. The system of claim 12 for delivering an electro-conductive solution into the pores of a recipient human skin under treatment, the system comprising:
a body electrode attached to the skin around a treatment area for nerve activation;
the electro-conductive solution; and
an electrobrush, wherein the electrobrush comprises a metal electrode and a silicon perforated tip,
wherein the electro-conductive solution flows on the perforated tip making contact with the metal electrode thereby closing an electrical circuit between the electrobrush and the body electrode through the nervous system situated therebetween and imparting said potential difference through the solution.

20. An apparatus for use in the system of claim 19 for delivering said electro-conductive solution into the pores of a recipient human skin under treatment, the apparatus comprising:
said electrobrush, the electrobrush comprising:
a tip having perforations;
a metal electrode;
a body having three portions—a front portion, a middle portion and a rear portion distal to the metal electrode;
a socket; and
a plug following the body; and
said body electrode attached to skin area around treatment area for nerve activation.
